(19) United States
(12) Patent Application Publication
(10) Pub. No.: US 2016/0000449 A1
(43) Pub. Date: Jan. 7, 2016

(54) STERILE READY-TO-USE SURGICAL TOOL AND ATTACHMENT SYSTEM

(71) Applicants: Peter M. Aman, Austin, TX (US); Richard Acevedo, Austin, TX (US)

(72) Inventors: Peter M. Aman, Austin, TX (US); Richard Acevedo, Austin, TX (US)

(21) Appl. No.: 14/746,841

(22) Filed: Jun. 23, 2015

Related U.S. Application Data
(60) Provisional application No. 62/020,387, filed on Jul. 2, 2014.

(51) Int. Cl. A61B 17/16; A61B 17/14

(52) U.S. Cl. CPC A61B 17/1628; A61B 17/148; A61B 17/1624; A61B 2017/00734

(57) ABSTRACT

A surgical tool and attachment system include a powered rotary driver, a power tool accessory for connection to the powered rotary driver, and an adapter. The adapter includes a first end and a second end. The first end includes means for attachment to a non-rotating portion of the power tool accessory. The second end includes a snap-in means for attachment to the powered rotary driver.

### CROSS REFERENCE TO RELATED APPLICATIONS

This application is related to and claims priority to U.S. Provisional Application No. 62/020,387, filed on Jul. 2, 2014, which is incorporated by reference herein in its' entirety.

### BACKGROUND

This disclosure relates to single and limited use surgical power tools and more specifically such tools which can be provided as a pre-packaged, pre-sterilized tool including a pre-installed charged battery.

Important factors for any surgical instrument include sterility, cost of acquisition, maintenance, and reliability during use in the surgical suite. Each of these factors can have a significant impact on the cost of medical care for both the patient and the provider.

In recent years, there has been significant focus on the ever increasing cost of medical care. These cost increases have led to skyrocketing insurance premiums, reduced coverage, reduced reimbursements, increased fees for services, severe reductions in services for some patient groups by some providers, and unfortunately an apparent increase in infections and medical mishaps.

In an effort to reduce costs and improve profitability, both service providers and medical device suppliers are continuously looking for ways to streamline procedures, cut time, cost, and risk from their products and services without reducing the quality of the products or services they provide to their customers. One area to benefit from these savings and improvements has been in the orthopedic surgical field through the use of high precision, battery powered surgical instrumentation. In the late 1960's and early 1970's, battery operated drills were bulky, ill-balanced and required multiple batteries to perform some surgeries due to the limited energy storage capacity and poor efficiency of the electric motors.

Since then, manufacturers have attempted to make batteries more efficient with higher energy storage capacity, reduced size, and improved rechargeable lifespans. Likewise, motor housings such as saw and drill bodies have become more ergonomic, balanced, lightweight and energy efficient. As with may standard hand tools having multiple moving components, instrument manufacturers have reduced weight by utilizing lighter materials such as plastic housings, and gears, and put lightening holes in what were previously solid housings. In some cases, standard mountings for attachments have been replaced with modular fittings, allowing for greater interchangeability and component selections. Additionally, manufacturers have attempted to improve electrical components by upgrading them with more modern components wherever possible.

All of these improvements in equipment construction have improved efficiencies, costs and quality in some areas while at the same time increasing costs for acquisition, maintenance and increasing risks in other ways that were not previously seen or predicted. Often times cost and quality can be inversely proportional to one another. One example of the increased cost and reduced patient safety is seen in the cleaning and maintenance of instruments.

Recent published reports suggest that many of the surgical instruments used in operations were not being cleaned and/or sterilized appropriately in the very hospital facilities that were established and tasked for that purpose. In numerous reports, following cleaning and sterilization, it was noted that upon closer secondary inspection, the inside of small diameter cannulas and intricate mini-components of arthroscopic shavers that are used for many of today's minimally invasive procedures, contained human tissue and bone fragments from previous surgeries. In other cases, modular components of drills and saws such as chucks, drill bits and blades were found to have similar debris or pieces of cleaning brushes and/or bristles embedded in or on them. These investigations have demonstrated that in most cases the instruments were not cleaned according to manufacturer's specifications which has likely lead to many documented cases of serious, multiple, serial infections for subsequent patients. A pilot program conducted by the Centers for Medicare and Medicaid Services (Schaefer et al., 2010; JAMA 2010; 303(22):2273-2279) inspected 1500 outpatient surgery centers and found that 28% had been cited for infectious control deficiencies associated with equipment cleaning and sterilization. The costs to the patients and the hospitals in both expense and liability to deal with these infections can be and has been staggering.

In other cases, critical battery-operated, motorized tools such as drills or bone saws have ceased to function due to dead batteries that no longer maintain their capacity to hold a charge, or due to internal part failure, often attributable to overuse or lack of proper maintenance. The resultant downtime in the operating suite is extremely costly, as the procedure step must be put on hold while replacement or substitute tools are obtained. Wait times may often exceed 20-30 minutes, resulting in additional anesthesia exposure for the patient, additional operating room time (charged to the patient) and potential delays to other procedures where the replacement or substitute equipment had been scheduled for use in a later procedure. Recent estimates (2005) establish the average cost of operating room time to range between $62/min. (range $21.80-$133.12) depending on the procedure. These figures did not include extra resources provided by the hospital for special, non-routine situations which often occur during standard procedures, and did not include the surgeon and anesthesia provider fees, (anesthesia fees are estimated to be $4/min; range $2.20-$6.10). In each case, the hospital or surgical center must then take the equipment out of service, and send it back to the instrument manufacturer or other service provider for service or replacement.

Hospitals and instrument manufacturers are continuously attempting to find improved ways to reduce risk associated with infection in general, and more recently, specifically from improperly cleaned instruments. One approach has been to use more disposable, single-use instruments such as drills, saw blades and plastic cannulas. Additionally, many laparoscopic devices such as, surgical staplers and trocars, are designed as single use items that are intended to be immediately disposed of after use. Unfortunately, at today's acquisition costs, the total cost of ownership and benefits are not always clear for high-use battery-operated, motorized instruments such as saws, drills and reamers used in orthopedic procedures and the idea of disposable powered instruments has not been readily embraced.

A recent trend in the medical community is reprocessing of single use medical instruments, by parties other than the original equipment manufacturer, instead of discarding them after use. During reprocessing, the medical instruments are disassembled, cleaned and sterilized. They are then reassembled for future use. However, because the medical instruments reprocessed for further use are specifically provided for use during a single procedure, the performance of the medical instruments tend to decline after reprocessing because the components making up the medical instrument are not adapted for multiple uses and will degrade in performance when used beyond their intended life span. For example, reprocessing of the cutting devices on trocars is intended to extend these devices beyond their intended mission life, but often results in duller cutting edges on the blades because neither the materials used nor the reprocessing method can restore the device to the original manufacturing specifications. A greater force, therefore, is needed to make an initial incision, causing more trauma to the patient. In addition, the use of greater force increases the potential for error during the surgical procedure.

Most hospitals and surgery centers buy high-use, reusable motorized, pneumatic, wired or battery operated, orthopedic surgical equipment and are expected to clean, sterilize, and maintain them internally within the hospital. Unfortunately, the technicians hired to perform this work are typically not qualified or trained to perform this work adequately for the many varieties of powered instruments used. Further, manufacturers rarely provide the hospital/client with the training or diagnostic equipment necessary to evaluate or test the equipment. Often times the hospital employees responsible for cleaning and maintenance are not technicians at all, being paid slightly more than minimum wage, working at a fast pace to merely wash, count, and reload instruments into their appropriate system trays and flash sterilize them as quickly as possible, in an effort to keep the equipment in rotation in the hospital operating rooms, where higher throughput dictates profitability for the hospital or surgery center.

As a result of high throughput requirements, general maintenance is rarely done and preventative monitoring and maintenance is almost never done on this type of equipment. Hospital budgets for internal maintenance of equipment are generally geared toward high-end, multi-million dollar capital equipment such as x-ray and radiological equipment. It is generally assumed that it is faster, simpler, and more economical for the hospital to wait for hand-held instruments, such as drills, saws and reamers to fail, then, send them back to the manufacturer for repair or replacement.

Thus it has become apparent that there is a need for an improved system of cost-effective, battery-operated, motorized tools in conjunction with better cleaning and maintenance protocols which can provide the hospital, surgeon, and most importantly, the patient, with a higher degree of efficiency and cleanliness while reducing risk and keeping the costs of cleaning, maintenance, and repair as low as possible.

### SUMMARY

A surgical tool and attachment system include a powered rotary driver, a power tool accessory for connection to the powered rotary driver, and an adapter. The adapter includes a first end and a second end. The first end includes means for attachment to a non-rotating portion of the power tool accessory. The second end includes a snap-in means for attachment to the powered rotary driver.

### BRIEF DESCRIPTION OF THE DRAWINGS

FIGS. 1 and 2 are perspective views illustrating an embodiment of a power driver, an accessory and an adapter in pre-assembled and assembled views, respectively.

FIGS. 3-6 are side views illustrating embodiments of single-use accessories including a wire driver, a Jacob's chuck and a reamer speed reducer, respectively.

### DETAILED DESCRIPTION

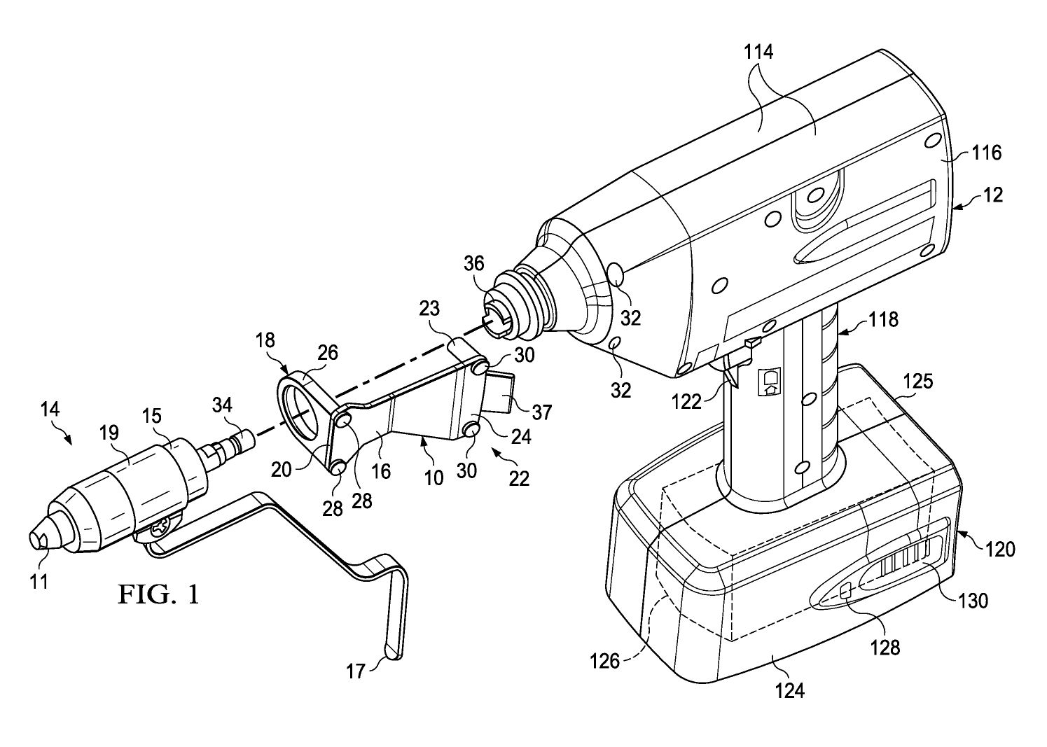

A medical procedure power tool such as a powered rotary driver hand tool 12 is illustrated in FIG. 1 and includes an integrated housing 114 having a power and attachment portion 116, a handle portion 118 and a battery housing portion 120. The power and attachment portion 116 contains an electric motor (not shown) and a chuck for securing various accessories to the tool 12. The handle portion 118 includes a trigger 122, and the battery-housing portion 120 defines a battery compartment 124. The integrated housing 114 is preferably formed of a synthetic material and comprises first and second halves. A battery 126 in battery compartment 124 may be either rechargeable or disposable. Battery housing portion 120 may include as an example, a single unit molded door 125 having a pair of snap-on attachment members 128 and a pair of grip-to-remove members 130.

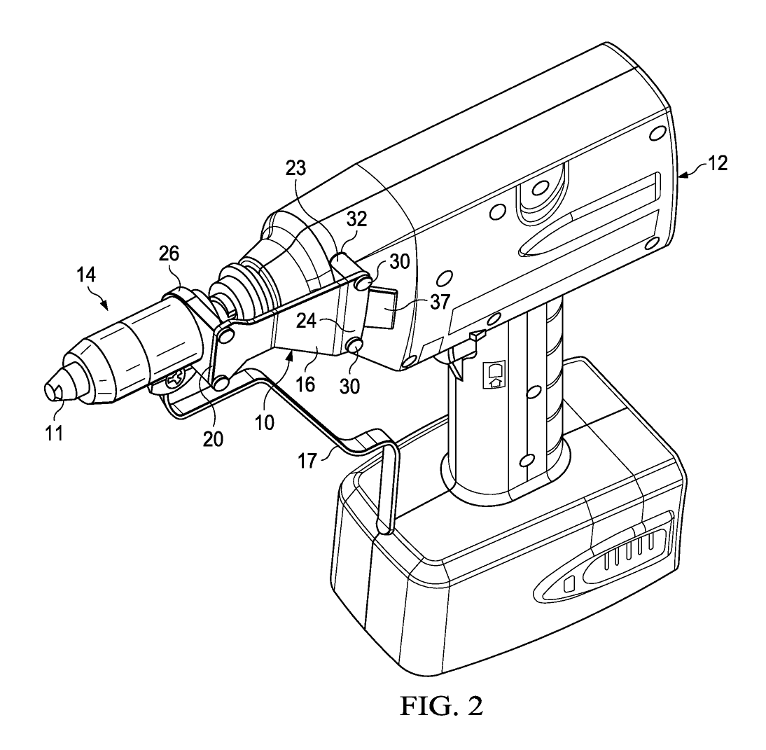

Surgical tools used for procedures such as wire or pin driving commonly use a complex system for attaching, for example, a wire and pin collet to a rotary handpiece. A device and system, see FIGS. 1 and 2, are provided to improve the commonly used coupling system. The device and system comprise a driver adapter 10 to be used to attach the powered rotary driver hand tool 12 to a wire driver accessory 14, as an example. The wire driver accessory 14 includes a flexible handgrip 17 and a clamp 19 attached to wire driver 14, for clamping and releasing wire or pins to be driven into human bone during a surgical procedure.

The driver adapter 10 preferably includes a flexible plate 16 formed of a suitable material having a first attachment 18 at a first end 20 of plate 16 for providing an adjustable clamping attachment to a non-rotating portion 15 of the wire driver 14. A second attachment 22 is positioned at a second end 24 of plate 16 for attachment to the powered rotary driver 12. However, the adapter 10 may also be a flexible bar, a hinged plate or a hanged bar, for example.

The first clamping attachment 18 includes a u-shaped clamp 26 and threaded attachment members 28 for securing the clamp 26 to the non-rotating portion 15 of the wire driver 14. The second attachment 22 includes a pair of locating pins 23 attached to the plate 16 by threaded attachment members 30. A plurality of apertures 32 are provided in powered rotary driver 12 to receive the locating pins 23. Only one of the locating pins 23 is visible in FIGS. 1 and 2.

The wire driver 14 includes a male coupling 34, such as a modified Hudson quick-connect, which inserts and locks in a female coupling 36 on the rotary driver 12 so that the driver 12 can drive a rotary element 11 of the wire driver 14. When the male coupling 34 is secured in the female coupling 36, locating pins 23 are positioned adjacent the apertures 32. The flexible plate 16 can be manually, if necessary, flexed by gripping a tab 37 adjacent the locating pins 23 so that the pins 23 can insert or snap-into the apertures 32, see the assembled view in FIG. 2.

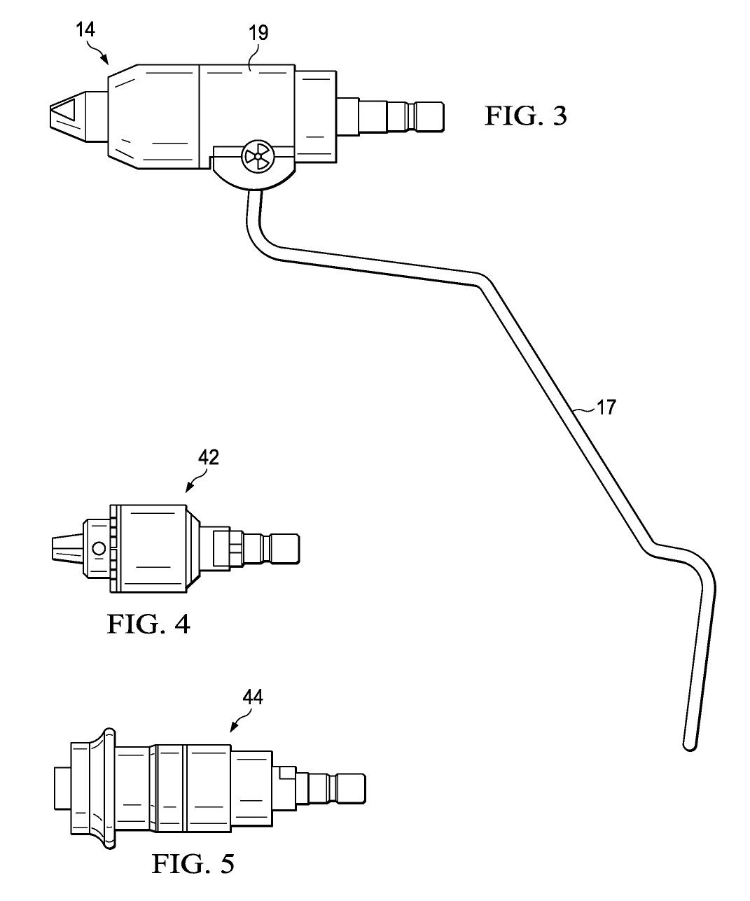

It is also contemplated that significant savings can be realized in providing for single-use accessories, see FIG. 3-5, such as wire driver 14, a Jacob's chuck 42, and a reamer speed reducer 44, by manufacturing such accessories having at least 50% of the components made of low cost injection molded components formed of a suitable synthetic material and providing selected remaining components to be made of metal where critically needed.

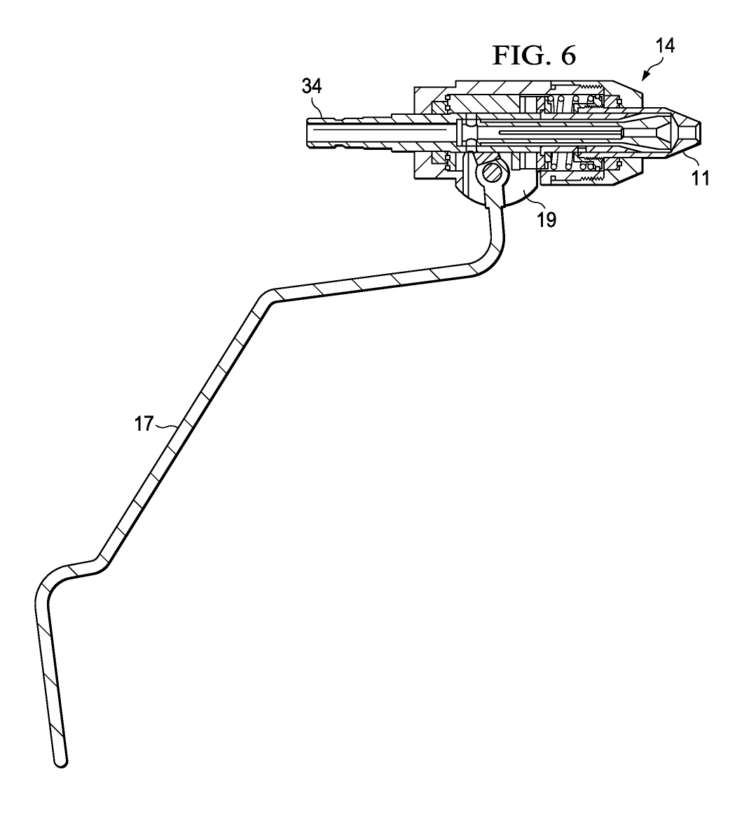

In FIG. 6, the wire driver 14 is illustrated in cross-section for the purpose of showing that a portion of the wire driver 14, up to 50%, can include components formed of a suitable synthetic material. In this example, the cross-hatch pattern is indicative of the metal portions of the wire driver 14, and also the handle 17, although the handle 17 can also be formed of a suitable synthetic material.

With the advent of single-use and limited use tools, utilizing a wire driver, chuck or the like that is traditionally high cost, machined stainless steel increases the cost and decreases the sterility advantages of a single or limited use tool. Utilizing a wire driver increases the cost of both the surgical tool and the wire driver. In order to attach a traditional wire driver to a tool, an expensive attachment scheme has been used which utilizes complex male and female interlocking components on the body of the rotary tool and also on the accessory. In order to maximize the benefits of a single or limited-use tool, a set of low cost, single or limited use accessories are needed. In order to reduce costs of both the tool and the wire driver, a lower cost attachment system is required.

A single-use or limited-use removable wire driver, composed primarily of plastic or other molded material can substantially reduce costs in comparison to the current iteration of the product which is primarily machined steel. The low cost wire driver would be disposable after a single use or a limited number of uses, not expected to be greater than 20 procedures. A key element of the disclosure is low cost, injection molded outer components combined with a minimal use of more robust metallic parts where necessary to maintain functional usefulness over the limited life of the device. This disclosure lowers the cost-per-procedure and minimizes sterility risks in comparison to utilizing traditional machined steel equivalents, especially when using the accessory in a single-use, disposable application.

A single-use or limited-use chuck is composed primarily of plastic or other molded material will substantially reduce costs in comparison to the current iteration of the product which is primarily machined steel. The low cost, removable chuck would be disposable after a single use or a limited number of uses, not expected to be greater than 20 procedures. A key element of the device and system is low cost, injection molded outer components combined with a minimal use of more robust metallic parts where necessary to maintain functional usefulness over the limited life of the device. This device and system lowers the cost-per-procedure and minimizes sterility risks in comparison to utilizing traditional machined steel equivalents, especially when using the attachment in a single-use, disposable application.

A single-use or limited-use Reamer Speed Reducer, which has a primary function to reduce RPM and increase torque of the rotating instrument, for powered surgical tools is composed primarily of plastic or other molded material to substantially reduce costs in comparison to the current iteration of the product which is primarily machined steel. The low cost, removable accessory would be disposable after a single use or a limited number of uses, not expected to be greater than 20 procedures. A key element of the device and system is low cost, injection molded outer components combined with a minimal use of more robust metallic parts where necessary to maintain functional usefulness over the limited life of the device. This device and system lowers the cost-per-procedure and minimizes sterility risks in comparison to utilizing traditional machined steel equivalents, especially when using the attachment in a single-use, disposable application.

A single-use or limited-use adapter, reamer shaft or other accessory for powered surgical tools are composed primarily of plastic or other molded material to substantially reduce costs in comparison to the current iteration of the product which is primarily machined steel. The low cost, removable adapter, reamer shaft or other accessory would be disposable after a single use or a limited number of uses, not expected to be greater than 20 procedures. A key element of the device is low cost, injection molded outer components combined with a minimal use of more robust metallic parts where necessary to maintain functional usefulness over the limited life of the device. This device and system lowers the cost-per-procedure and minimizes sterility risks in comparison to utilizing traditional machined steel equivalents, especially when using the accessory in a single-use, disposable application.

The device and system utilizes a simple, low cost attachment system for a wire driver and other accessories commonly used with powered surgical instruments. Surgical power tools and associated accessories currently utilize the complex attachment scheme mentioned above, which is a holding device, specifically, a subtype of chuck that forms a collar around the object to be held and exerts a strong clamping force on the object when it is tightened, usually by means of a tapered outer collar. This system requires multiple, expensive components that drive up the cost of the power tool as well as the attachment. The device removes the necessity of the complex attachment scheme for certain accessories such as a wire driver, removing cost from both the power tool and the accessory.

The device and system uses a male/female coupling on the wire driver and power tool to attach the rotating mechanism, combined with an adapter plate with locating pins which eliminates any clockwise or counterclockwise rotation of the wire driver when the surgical drill shaft is rotating. The adapter plate can be modified to attach to the right, left or both sides of the tool, as well as modifying pin locations depending on the tool housing structure.

Although illustrative embodiments have been shown and described, a wide range of modification, change and substitution is contemplated in the foregoing disclosure and in some instances, some features of the embodiments may be employed without a corresponding use of other features. Accordingly, it is appropriate that the appended claims be construed broadly and in a manner consistent with the scope of the embodiments disclosed herein.

What is claimed is:

1. A surgical tool attachment system comprising:
a powered rotary driver;
a power tool accessory means for connecting to the powered rotary driver; and
an adapter having a first end and a second end, the first end including means for attachment to a non-rotating portion of the power tool accessory means, and the second end including a snap-in means for attachment to the powered rotary driver.

2. The tool of claim 1 including an integrated housing.

3. The tool of claim 2 wherein the integrated housing includes a power and attachment portion, a handle portion and a battery-housing portion.

4. The tool of claim 3 wherein the battery-housing portion includes a rechargeable battery.

5. The tool of claim 3 wherein the battery-housing portion includes a disposable battery.

6. The tool of claim 3 wherein a removable battery is mounted in the battery-housing portion.

7. The tool of claim 1 wherein the means for attachment to a non-rotating portion of the power tool accessory means is a clamp attached to a first end of the adapter.

8. The tool of claim 7 wherein the snap-in means includes at least one locating pin attached to a second end of the adapter, opposite the first end.

9. The tool of claim 8 further comprising:
at least one aperture provided in the powered rotary driver for receiving the at least one locating pin.

10. The tool of claim 1, further comprising:
a manually grippable tab adjacent the second end of the adapter which enables flexing movement to be manually imposed on the adapter.

11. The tool of claim 1, further comprising:
a flexible handle having a first end connected to the power tool accessory means, the handle having a terminal end positioned adjacent the powered rotary driver.

12. The system of claim 1 wherein the power tool accessory means includes a portion of components being formed of a suitable synthetic molded material and a portion of components formed of a metal material, the synthetic material comprising at least 50% of the total material of the power tool accessory.

13. A surgical tool attachment system comprising:
a powered rotary driver;
a power tool accessory means for connecting to the powered rotary driver; and
wherein the power tool accessory means includes a portion of components being formed of a suitable synthetic molded material and a portion of components formed of a metal material, the synthetic material comprising at least 50% of the total material of the power tool accessory.

14. A surgical tool attachment method comprising:
providing a powered rotary driver;
attaching a power tool accessory to the powered rotary driver by means of an adapter including a first end and a second end;
attaching the first end of the adapter to a non-rotating portion of the power tool accessory; and
attaching the second end of the adapter to the powered rotary driver by means of a snap-in attachment.

15. The method of claim 14 wherein the first end of the adapter is attached to the power tool accessory by a clamp.

16. The method of claim 14 wherein the snap-in attachment includes at least one locating pin attached to the second end of the adapter, and a locating pin receiving aperture provided in the powered rotary driver.

17. The method of claim 14, further comprising:
manually flexing the adapter by means of a manually grippable tab extending from the second end of the adapter.

18. The method of claim 14, further comprising:
attaching a first end of a flexible handle to the power tool accessory, whereby a terminal end of the flexible handle is positioned adjacent the powered rotary driver.

19. The method of claim 18 wherein the first end of the flexible handle is attached to a non-rotating portion of the power tool accessory.

20. The method of claim 14 wherein the power tool accessory includes a portion of components formed of a synthetic material and a portion of components formed of a metal material, the synthetic material comprising at least 50% of the total material of the power tool accessory.
